(19) United States
(12) Patent Application Publication
(10) Pub. No.: US 2010/0087769 A1
(43) Pub. Date: Apr. 8, 2010

(54) BIOCIDIC MEDICAL DEVICES, IMPLANTS AND WOUND DRESSINGS

(75) Inventors: Shmuel Bukshpan, Ramat Ha-Sharon (IL); Gleb Zilberstein, Rechovot (IL)

Correspondence Address: The Law Office of Michael E. Kondoudis, 888 16th Street, N.W., Suite 800, Washington, DC, 20006, US

(73) Assignee: OPLON B.V., Delft (NL)

(21) Appl. No.: 12/598,429

(22) PCT Filed: Apr. 3, 2008
(86) PCT No.: PCT/IL2008/000467

§ 371 Date: Nov. 18, 2009

Related U.S. Application Data
(60) Provisional application No. 60/924,154, filed on May 1, 2007.

(51) Int. Cl. A61F 13/00; A61L 27/50; A61L 15/42; A61L 2/00

(52) U.S. Cl. 602/48; 422/28

(57) ABSTRACT

The present invention discloses a medical device selected from a group consisting of medical devices, implants wound dressings, comprises at least one insoluble proton sink or source (PSS). The medical device is provided useful for killing living target cells (LTCs), or otherwise disrupting vital intracellular processes and/or intercellular interactions of the LTC upon contact. The PSS comprises, inter alia, (i) proton source or sink providing a buffering capacity; and (ii) means providing proton conductivity and/or electrical potential. The PSS is effectively disrupting the pH homeostasis and/or electrical balance within the confined volume of the LTC and/or disrupting vital intercellular interactions of the LTCs while efficiently preserving the pH of the LTCs' environment.

### FIELD OF THE INVENTION

The present invention pertains to medical devices, implants and wound dressings. More specifically, the invention relates to biocidic medical devices, implants and wound dressings which comprise means for killing living target cells, or otherwise disrupting vital intracellular processes and/or intercellular interactions of said cells upon contact.

### BACKGROUND OF THE INVENTION

It is well known in the art that medical devices, implants and wound dressings which comprises means for killing living target cells, or otherwise disrupting vital intracellular processes and/or intercellular interactions of said cells upon contact, while efficiently preserving the pH and other life-effecting parameters of the cell's environment, are a long felt need. For sack of clarification, the background will first focus the medical devices industry, and will than approach the wound dressing industry.

Medical Devices

Biofilms can colonize almost all surfaces, from glass to steel, from cellulose to silicone, which are the main materials used to produce medical devices. For fifty years, medical instruments have been sterilized by the medical industry with gaseous agents such as ethylene oxide and chlorine dioxide, which are commercialized in nonflammable blends with inert carrier gases to overcome their explosive character [1]. However, many of the corrosion or fouling processes which take place after adhesion and growth of micro-organisms occur inside the human body. Harsh treatments of the surfaces of the devices to prevent and/or destroy cell adhesion are, therefore, hampered.

Protective coatings have been the most wide spread method to prevent corrosion in metallic surfaces. However, cathodic protection appears as a good protective technique, especially in the medical field. Cathodic protection acts by providing an electrical current from external sources to counteract the normal electrochemical corrosion reactions.

Anti-corrosion and anti-fouling, combined with anti-rejection and antibiotic properties of medical devices for intra and extra body application were achieved by placing a semi-conductive coating in metallic devices [2]. The technique uses semiconductor technology with no external anode, electrolyte or current flow. An electronic filter, connected to the conductive coating, monitors and minimizes the corrosive noise generated by the coated conductive structure.

Other invention uses “oligodynamic iontophoresis” to prevent microbial adhesion to intraocular lens [3]. This technique involves the movement of ions from a metal such as silver to a conductive medium (saline, blood or urine) by application of an electrical current. Silver is effective against a broad range of bacterial, yeast and fungal cells since the positively charged silver ions can interact with thiol groups of membrane-bound enzymes and proteins, uncouple the respiratory chain from oxidative phosphorylation or collapse the proton-driving force across the cytoplasmic membrane [4,5]. The current required to remove a bactericidal amount of silver ions from electrodes into solution is in the range of 1-400 mAmps. Since an external electric power supply is required, oligodynamic iontophoresis has had limited use in medical devices. However, a composite material made of a conductive organic polymer matrix, in which two metals with a chemical half-cell potential difference are suspended, can act as an iontophoretic material [6]. The voltage potential generated between the two dissimilar metals generates a current of electrons in the conductive matrix after exposure to an electrolyte solution such as body fluids.

An improvement to an implantable port and other devices such as pacemakers and artificial joins, also encompass the presence of metallic silver, an inorganic silver compound, a silver salt of an organic acid or other antimicrobial compounds as taurolidine on the surface of the port or device [7]. The improved implantable port, including a housing, contains a silver coated surface and is implanted within a subcutaneous tissue pocket, or uses a separate container, in the form of a pouch, that is placed over the device before implantation in the subcutaneous pocket or used as a reservoir to hold the antimicrobial solution (e.g. taurolidine).

Biodegradable microshapes, such as microspheres, containing time-release agents effective against bacterial biofilms can be placed into the gingival crevice or periodontal region to combat bacteria adhesion to teeth [9].

Bacterial plaque is the main cause of several periodontal diseases, including gingivitis and periodontitis. This technique may overcome the difficulties of periodontal prevention and therapy based on the individual motivation and skill to use toothbrushes, dental floss and other oral hygiene instruments.

Bacterial interactions, which may be synergistic or antagonistic, have a major role in maintaining the flora of skin, intestines, uroepithelial cells and mucous membranes, and thus in preventing the establishment of pathogenic bacteria. Several mechanisms of bacteria interference have been described, e.g., production of antagonistic substances, competition of nutrients, changes in the microenvironment and lack of available adhesion area for the pathogenic bacteria due to the presence of the non-pathogenic strains [10]. A recent patent describes how an antimicrobial and a non-pathogenic bacterial coating layer may be effective to demote the infection of surfaces by pathogenic microorganisms [11]. The non-pathogenic bacteria, resistant to the antimicrobial used, should interfere with pathogenic strains trying to colonize the surface and dominate the ecological space. The antimicrobial agent, to be used with a kit applied to the medical device before implantation, can be an antibiotic, an antiseptic, a disinfectant or a combination of the three. Non-pathogenic gram-negative bacterium should be selected from Enterobacteriacea (e.g. Escherichia, Salmonella and Yersinia), Pseudomonas aeruginosa, Stenotrophomonas maltophilia, Burkholderia cepacia, Gardnerella vaginalis and Acinetobacter species. In the patent's context, “non-pathogenic bacteria” referrers to known non-pathogenic bacteria and pathogenic bacteria that have been mutated or converted to non-pathogenic strains.

Biofilm Prevention in Catheters: Catheters for vascular access and haemodynamic monitoring (e.g. infusion of electrolytes, drugs or chemotherapy agents; draw of blood for analysis and haemodialysis) are one of the most used types of medical devices and responsible for a high number of nosocomial infections [12]. There are four potential sources of catheter infection: (i) the presence of microbial cells at the site where the catheter is inserted through the skin; (ii) the catheter hub; (iii) pathogenic cells traveling through the blood stream from a distant infection site; (iv) contamination of the infusion fluid [13]. The degree of pathogenicity will depend upon microbial adherence, which is also related to the catheter material and the host defense system.

Peritoneal dialysis catheters for acute use (application for less than 4 days) are usually made of relatively rigid nylon or polyethylene, whilst those for chronic afflictions are fabricated with soft materials, such as silicone rubber or polyurethane. The chronic catheters have extra-peritoneal cuffs that cause local inflammation and tissue growth; that helps positioning the catheter while prevents fluid leaks and bacterial colonization. Nevertheless, since silicone is a hydrophobic polymer, it is susceptible to biofilm formation.

However, the hydrophilic polyurethane has also been reported to be attacked by micro-organisms. An antimicrobial agent such as triclosan or butyl paraben can be dispersed in medical grade silicone elastomer used to fabricate prosthetics and parts of voice prosthesis [14].

During peritoneal dialysis, the catheter is introduced directly in the peritoneal cavity of the patient and the catabolites migrate from the blood, across the peritoneal membrane, to the dialysis solution. An expected complication of peritoneal dialysis is peritonitis, being the catheter the major access for infection as it makes the bridge between the sterile inside the peritoneal cavity and the exterior of the body. Addition of 0.5-4% taurolidine into peritoneal dialysis solutions, and to lock and flushing solutions, may reduce or prevent microbial colonization [15].

Besides risk of infection, clotting of catheters may also occur, since these devices may rest in the patient's body for a significant time, being used on a weekly or daily basis. To prevent the formation of thrombus, catheters are usually filled with a lock solution. The anticoagulant normally applied is heparin, which is injected into each catheter lumen immediately after each use. The heparin solution should be maintained in the lumen but must be withdrawn before the next application because heparin may cause haemorrhages.

Several innovations have been presented related to the lock solution. In one method, a syringe containing a lock solution of citrate salt (1.5-50%, w/w) is used to infuse the lumen of an indwelling catheter [16]. As alternatives, polyethylene glycol, glycerol, polyglicerol, polygeline or mixtures of them, may be added to the lock solution to increase its viscosity and density to expand the time of permanency, or the lock solution may be prepared to have a pH lower than 6.5.

A method to treat infections related to indwelling catheters was developed based on the application of an electric field through two electrodes applied to the internal and external surfaces of the catheter [17]. An antimicrobial drug solution is inserted in the catheter and in the receptacle of the electrode placed on the skin and around the exit area of the catheter. The permeability of the catheter to antimicrobial drugs increases both in the internal and external surfaces, helping to kill micro-organisms in difficult to access places.

Crossley proposed a technique that uses photodynamic therapy: light of a selected wavelength or wavelength band is coupled to the medical device and activates the release of a toxic substance from at least one photosensitizer compound embedded in the surface [18].

The photosensitizer may be a natural compound (such as porphyrins, polyynes or anthraquinones), a dye (rhodamines, methylene blue, etc) or other substance that reacts to light (such as cyanine compounds). Antimicrobial activity of these compounds may be inherent or acquired upon exposure to light.

Antimicrobial substances required to destroy biofilms are not only toxic to micro-organisms but may also be toxic to the patient, causing allergic reactions, whilst some microorganisms may produce specific compounds able to destroy the biocide molecule. To overcome these disadvantages, biocompatible acid precursors may be used to inhibit microbial attachment and/or growth on the surface of medical devices [8]. This is achieved once the device is placed inside the patient's body, as acid moieties are produced from the acid precursor (examples include glycolide, lactide, p-dioxanone, glycyl glycolate and lactyl lactate), lowering the pH of the coating and adjacent device.

The acid precursor may (i) diffuse through the coating and hydrolyse at the surface or (ii) first hydrolyse and the resulting acid diffuse through the coating to the surface.

U.S. Pat. No. 6,514,517 describe compositions containing a biocompatible acid precursor in amounts effective to inhibit microbial attachment and/or growth on a surface of a medical device having the composition applied thereto, to coatings or films prepared from such compositions and to medical devices having the composition applied to a surface thereof. Once the coated medical device is placed in the body of a mammal, e.g. a human or animal, the acid precursor in the coating produces acid moieties at concentrations effective to maintain the pH of the coating and/or the tissue area immediate to and adjacent the device at a level effective to inhibit microbial attachment and/or growth on the coated surface of the medical device. The acid precursor may diffuse through the coating and then hydrolyze at the surface of the coated device, or in the immediate vicinity. Alternately, or in combination with the above, upon implantation of the coated device, the acid precursor may first hydrolyze, with the resulting acid diffusing through the coating to the surface to provide the effective pH.

Similarly, U.S. Pat. Nos. 6,514,517 and 5,820,607 describes a general concept of an indwelling medical devices constructed from permeable or non-permeable material having a pharmacologically active ingredient layer surrounding the device, and an outer sheath which is permeable to the pharmacologically active ingredient. This construction provides a device that allows the pharmacologically active ingredient located between the catheter tube and the outer sheath to slowly diffuse through the outer sheath and/or inner tube, thus inhibiting microbial infection on the outer surface and lumen of the catheter.

US patent application 2003/0147960 describe an ionic antimicrobial coating which contain a water-insoluble polymer having a first ionized group and an antimicrobial agent having a second ionized group with a charge opposite to that of the first ionized group. The antimicrobial agent is attached to the water-insoluble polymer via an ionic bond between the first ionized group and the second ionized group thereby providing a medical device (e.g. catheter) having a sustained-release depot of an antimicrobial agent (silver chloride).

However, as oppose to the above described patent applications, the compositions and materials of the current invention are non-soluble and non-diffusible but rather solid ion-exchange materials which are not susceptible to saturation or depletion and possess a long acting characteristics. Moreover, the compositions and materials of the current invention are by themselves, inherently antimicrobial without the addition or bounding of any external agent.

Wound Dressing

Several studies have demonstrated the role of topical antimicrobials in decreasing morbidity and mortality in patients with major skin injuries (partial- or full-thickness skin involvement), particularly before early excision (24, 33, 35). A recent study conducted by the U.S. Army Burn Center compared the levels of mortality of adult patients according to age and burn size before (1950 to 1963) and after (1964 to 1968) the introduction of mafenide acetate topical antibiotic therapy (27). Use of mafenide acetate was associated with a greater than 10% reduction in mortality for those with burns of 40 to 79% TBSA, but its use had only a minimal effect on mortality in patients with smaller or much larger burn injuries. The efficacy of various topical antimicrobials in common use in wound care and treatment is dynamic due to the ability of microorganisms to develop resistance rapidly (20). The sustained potency of individual agents depends on the extent of use and the resident nosocomial flora within any specialized wound treatment center.

Topical Antimicrobial Therapy Widespread application of an effective topical antimicrobial agent substantially reduces the microbial load on the open wound surface and reduces the risk of infection (42, 51, and 53). By controlling infection, effective topical antimicrobial therapy decreases the conversion of partial-thickness to full-thickness wounds, but its use is adjunctive to early excision therapy. Selection of topical antimicrobial therapy should be based on the agent's ability to inhibit the microorganisms recovered from wound surveillance cultures and monitoring of the nosocomial infections acquired in the wound treatment unit. Prescription is also based on the individual preparation of the topical agent (e.g., ointment or cream versus solution or dressing) and its pharmacokinetic properties. Wound units may rotate the use of various topical antimicrobial preparations on a regular basis to decrease the potential for development of antibiotic resistance (20, 33, and 54). Topical antibiotic agents should first be applied directly to the patient's dressings before application to the wound to prevent contamination of the agent's container by wound flora.

Table 1 outlines the most widely used topical antimicrobial agents and new silver nanocrystalline dressings that are based on the bactericidal properties of the silver ion (35, 42, and 51). The inhibitory action of silver is due to its strong interaction with thiol groups present in the respiratory enzymes in the bacterial cell (46, 47). Silver has also been shown to interact with structural proteins and preferentially bind with DNA bases to inhibit replication (45, 46). For this reason, silver has recently been shown to be highly toxic to keratinocytes and fibroblasts and may delay wound healing if applied indiscriminately to debrided healing tissue areas (26, 31, and 45). Moist exposure therapy using a moisture-retentive ointment (MEBO-Julphar; Gulf Pharmaceutical Industries, United Arab Emirates) has recently been shown to act as an effective antibacterial agent while promoting rapid autolysis debridement and optimal moist wound healing in partial-thickness injury (21, 23). Moisture-retentive ointment also resulted in earlier recovery of keratinocytes with improved wound healing and decreased scar formation (22). The topical antimicrobial agents reviewed are primarily used in burn/wound center patients with full-thickness or deep partial-thickness burn wounds.

Silver nitrate Silver nitrate is rarely used nowadays in modern burn/wound units because the deposition of silver discolors the wound bed and other topical agents are available that are easier to use and have less potential toxicity. Silver nitrate is most effective before the wound becomes colonized. The wound needs to be cleansed of emollients and other debris before a multilayered dressing is applied to the wound and subsequently saturated with silver nitrate solution. Effective use of this preparation therefore requires continuous application with secondary occlusive dressings, making examination of the wound difficult. The silver ion in AgNO₃ also quickly binds to elemental chlorine ions, so that repeated or large-surface application of this solution may lead to electrolyte imbalance (e.g., hyponatremia and hypochloremia) (42, 51). Silver nitrate antibacterial activity is limited to the wound surface (44, 59). This agent demonstrates bacteriostatic activity against gram-negative aerobic bacteria such as Pseudomonas aeruginosa and Escherichia coli, but it is not active against other genera, including Klebsiella, Providencia, and Enterobacter (42, 48). Silver nitrate also has limited antifungal activity, so that nystatin should be used concomitantly (43, 62).

Silver sulfadiazine Silver sulfadiazine is the most commonly used topical antibiotic agent for both ambulatory and hospitalized patients. This agent is a combination of sodium sulfadiazine and silver nitrate. The silver ion binds to the microorganism's nucleic acid, releasing the sulfadiazine, which then interferes with the metabolism of the microbe (46). It is easy to use and painless when applied and can be used with or without a dressing. Limited systemic toxicity with repeated daily or twice-daily application has occurred aside from the development of leukopenia (28, 47). Silver sulfadiazine has excellent broad spectrum antibacterial coverage against Pseudomonas aeruginosa and other gram-negative enteric bacteria, although some resistance has recently been reported (42, 56). This agent also has some activity against Candida albicans, but enhanced antifungal activity can be achieved by using nystatin in combination with silver sulfadiazine (43, 62). Although silver sulfadiazine dissociates more slowly than silver nitrate, there is still poor penetration into the wound (44, 59). Silver sulfadiazine is only absorbed within the surface epidermal layer, which limits its effectiveness in some patients with severe injuries. In Europe, a combination of cerium nitrate and silver sulfadiazine (Flammacerium; Solvay Duphar, The Netherlands) has been used to combat this problem (38, 39). Flammacerium has been shown to reduce the inflammatory response to injury, decrease bacterial colonization, and provide a firm Eschar for easier wound management (39).

Mafenide acetate: Topical mafenide acetate cream allows open wound therapy and regular examination of the wound surface because it is used without dressings. The wound surface is cleansed of debris prior to application of the cream, and the treated wound surface is left exposed after the cream is applied for maximal antimicrobial effect. Mafenide acetate is applied a minimum of twice daily and has been shown to penetrate the wound Eschar (59). The 5% solution must be applied to saturate gauze dressings, and these need to be changed every 8 hours for maximal effect. Mafenide acetate solution appears to be as effective as the cream preparation when used in this way (36, 42). Mafenide acetate (Sulfamylon) cream has a broad spectrum of activity against gram-negative bacteria, particularly Pseudomonas aeruginosa, but has little activity against gram-positive aerobic bacteria such as Staphylococcus aureus (42). This agent also inhibits anaerobes such as Clostridium spp.

Because protracted use of mafenide acetate favors the overgrowth of Candida albicans and other fungi, this agent should be used in combination with nystatin to prevent this complication due to its limited antifungal activity (43, 62). Despite its antibacterial potency, mafenide acetate is not as widely used as other agents because of its toxicity profile. This compound is converted to p-sulfamylvanzoic acid by monoamide oxidase, a carbonic anhydrase inhibitor, causing metabolic acidosis in the wound patient (42, 51). In wound patients with inhalation injury and a concomitant respiratory acidosis, the use of mafenide acetate over a large wound surface area or the repeated application of this compound can be fatal. Mafenide acetate also decreases the breaking strength of healed wounds and delays healing (26).

TABLE 1
Profile of commonly used topical antimicrobial agents ^{a}

| | | Eschar | | Major |
|---|---|---|---|---|
| Topical agent | Preparation | penetration | Antibacterial activity ^{b} | toxicity |
| Silver nitrate | 0.5% solution | None | Bacteriostatic against | Electrolyte |
| (AgNO₃) | | | aerobic gram-negative | imbalance |
| | | | bacilli, P. aeruginosa, | |
| | | | limited antifungal | |
| Silver | 1% water-soluble | None | Bactericidal against | Leukopenia |
| sulfadiazine | cream (oil-in-water | | aerobic gram-negative | |
| (Silvodene, | emulsion) | | bacilli, P. aeruginosa, | |
| Flamazine, | | | some C. albicans | |
| Thermazine, | | | | |
| Burnazine) | | | | |
| Mafenide acetate | 10% water-soluble | Limited | Broad spectrum against | Metabolic |
| (Sulfamylon) | cream (oil-in-water | | aerobic gram-negative | acidosis |
| | emulsion), | | bacilli, P. aeruginosa, | |
| | 5% solution | | anaerobes | |
| Nanocrystalline | Dressing | Moderate | Potent activity against | Limited |
| silver dressings | consisting of two | | aerobic gram-negative | toxicity |
| (Acticoat A.B. | sheets of | | bacilli, P. aeruginosa, | |
| dressing, | highdensity | | aerobic gram-positive | |
| Silverlon | polyethylene mesh | | bacilli, MRSA, VRE, | |
| | coated with | | multidrug-resistant | |
| | nanocrystalline | | Enterobacteriaceae | |
| | silver | | | |

^{a} Data are from references 35, 42, and 51
^{b} VRE, vancomycin-resistant enterococci.

Acticoat A.B. dressing/Silverlon. This product is a specialized dressing that consists of two sheets of high-density polyethylene mesh coated with nanocrystalline silver (e.g., ionic silver with a rayon-polyester core) (32, 61, and 63). The more controlled and prolonged release of nanocrystalline silver to the wound area allows less-frequent dressing changes, reducing the risk of tissue damage, nosocomial infection, patient discomfort, and the overall cost of topical therapy (32, 41). Nanocrystalline dressings may also provide better penetration of unexcised wounds because of their prolonged mechanism of action. Acticoat A.B. dressing with SILCRYST⁺ (Smith & Nephew Wound Management, Largo, Fla.), Silverlon (Argentum Medical, L.L.C., Lakemont, Ga.), and Silvasorb (Medline Industries Inc., Mundelein, Ill.) provides the most comprehensive broad-spectrum bactericidal coverage against important wound pathogens of any topical antimicrobial preparation currently marketed (32, 41). These dressings have potent antibacterial activity against most aerobic gram negatives, including Pseudomonas aeruginosa and antibioticresistant members of the family Enterobacteriaceae as well as aerobic gram positive bacteria, including MRSA and vancomycin-resistant enterococci (32, 41, and 63). If the wound surface has minimal exudates, these specialized dressings can remain in place for several days and retain antibacterial activity (41). This approach is replacing the use of other silver-based topical antibiotics in many wound centers.

Mupirocin (Bactroban) Mupirocin (pseudomonic acid A) is a fermentation product of Pseudomonas fluorescens (42, 55). This antibiotic has potent inhibitory activity against gram-positive skin flora such as coagulase-negative staphylococci and Staphylococcus aureus, including MRSA (49, 57, 58, and 60). Although primarily marketed for nasal decontamination, mupirocin has increasingly been used as a wound topical agent in North America, where MRSA has become a problem (34, 49). Mupirocin is currently not licensed in Europe for use as a topical agent. Various topical antibiotic preparations, including 1% silver sulfadiazine, 2% mupirocin, and 2% fusidic acid, were recently compared for their antibacterial effect in an MRSA-infected full-skin-thickness rat wound model (19). All of these agents were found to be equally effective against MRSA in reducing local wound bacterial counts and preventing systemic infection. Wound and burn treatment centers where MRSA is a problem may therefore rotate the use of topical mupirocin in combination with these other agents in order to decrease the development of resistance.

Nystatin Nystatin, (Mycostatin or Nilstat) is produced by Streptomyces noursei and has potent antifungal effect by binding to the sterols in the fungal cell membrane (42, 55). A lower concentration (3 μg/ml) of this agent inhibits Candida albicans, but a higher concentration (6.25 μg/ml) is needed to inhibit other Candida spp. and fungi (37, 52). A recent study of nystatin powder at a concentration of 6 million units/g showed that this approach was effective in treating four burn patients with severe angioinvasive fungal infections due to either Aspergillus or Fusarium spp. (49). Both superficial and deep-tissue wound infections were eradicated using nystatin powder without any other interventions or adverse effects on wound healing (49, 52). However, since nystatin has no activity against bacteria, it should be used in combination with a topical agent that has activity against the broad spectrum of pathogenic bacteria that cause wound colonization and infection (42).

Other topical antimicrobials Several other topical antimicrobials have also been used for topical wound therapy, including gentamicin sulfate (0.1% water-soluble cream), betadine (10% povidone-iodine ointment), bacitracin-polymyxin ointment, and nitrofurantoin (42, 55). However, these compounds are no longer used extensively because significant resistance has developed and/or they have been shown to be toxic or ineffective at controlling localized wound infections. Topical bacitracin-polymyxin is primarily used as a non-adherent, nontoxic petroleum-based ointment for skin graft dressings and for dressing partial-thickness wounds, particularly in children (55).

Medwrap™ (cf. U.S. Pat. No. 6,168,800) Island wound dressing with Microban® antimicrobial product (2,4,4′-trichloro-2′ hydroxyphenyl ether, also known as triclosan) provides a multilayer design barrier that maintains a moist environment for optimal healing and inside the dressing built-in Microban antimicrobial protection inhibits the growth of a broad spectrum of bacteria, including Staphylococcus aueus, MRSA and VRE, on the critical surface layer of the dressing. The Medwrap™ Island Wound Dressing with Microban is available in a variety of sizes and is medical device regulated by the FDA.

As the development of bacterial resistance to antibiotics continues and controversy regarding the use of topical antiseptics persists, the need for identification and development of new antimicrobial agents that are safe and broadly effective and have a low propensity to induce resistance becomes increasingly critical. In recent years, widespread interest has focused on a class of naturally occurring peptides that protect a variety of animals from infection. These peptides are found in a variety of cell types and operate by attaching to microbial cells, perforating the cell wall, and inducing leakage of cell contents. Such pore-forming antimicrobial peptides are widespread throughout nature: human neutrophils produce defensins, magainins have been isolated from the skin of the African clawed frog, and cecropins have a similar function in the giant silkworm moth (29). The opportunity to synthesize more potent and broad-spectrum analogues of the natural endogenous peptides has been recognized by pharmaceutical companies, and topical formulations are now in development for indications such as infected diabetic foot ulcers (40).

Concern over the use of antibiotics and the search for new antimicrobial agents has also led to the reemergence of therapies that have been used for centuries but have become less fashionable during the antibiotic era. However, despite the potential for novel agents such as tea tree oil, their acceptance and use in wound management will be limited until adequate safety and clinical efficacy data have been generated.

Honey is another ancient remedy that is gaining renewed popularity as alternative treatments for antibiotic-resistant bacteria are pursued. The observed benefits of honey in infected wounds may also be attributed to the high glucose content and low pH, both of which are stimulatory to macrophages (40).

Despite the multifactorial benefits of certain types of honey in the management of many wound types, widespread acceptability is likely to be slow at best. This assumption is based on the fact that such therapy is ancient and therefore represents a regressive step rather than advancing toward new and innovative therapies, and it is also based on the wide variation in potency that exists in honeys derived from different floral sources (50).

PCT application WO00/24378, to Ritter V. and Ritter M. and references therein, describe and teaches the use of polymeric microspheres having a substantial surface charge, either alone or as carriers of pharmaceutical agent for application to wound healing and/or lesions. PCT WO 2005/115336 to Hirsh M., et al., describes the use of binding resins, such as ion-exchange resins to allow drugs with incompatible solvent requirements to be prepared in a single-phase formulation for topical spray or foam wherein at least one of the drugs is bound to an ion-exchange resin. US patent application 2003/0147960 to Lin et al., describe ionic antimicrobial coating for application in medical devices that contain a water-insoluble polymer having a first ionized group and an antimicrobial agent having a second ionized group with a charge opposite to that of the first ionized group, in which the antimicrobial agent is attached to the water-insoluble polymer via an ionic bond between the first ionized group and the second ionized group. This composition provides a prolonged drug release and antimicrobial activity that is controlled by an ion-exchange mechanism. The antimicrobial agent was a chloride salt of silver. Therefore, the antimicrobial activity was the result of the sliver ions and not due to the ion-exchange properties of the polymer which serve here as a drug depot.

U.S. Pat. No. 6,800,278 to Perault et al., describes a composition and method for treating a wound with an inherently antimicrobial dressing. The dressing is a hydrogel containing from about 15 to 95 percent, and preferably from about 61 to 90 percent, by weight of a cationic quaternary amine acrylate polymer prepared by the polymerization of acryloyloxyethyl(or propyl)-trialkyl(or aryl)-substituted ammonium salts or acrylamidoethyl(or propyl)-trialkyl(or aryl)-substituted ammonium salts. The antimicrobial hydrogels are non-irritating to the wound, absorb wound exudates, and, due to the inherently antimicrobial properties, enhance the sterile environment around the wound. If desired, additional antimicrobial or other pharmaceutically active agents can also be incorporated into the hydrogel structure.

The following publications are hence incorporated as reference for the present invention: [19 Aguilera, A. M., Bitney, R. G., Conviser, S. A., Decaire, B. R.: US20036605254 (2003). [29 Bonaventura, J., Ignarro, L., Dowling, D. B., Spivack, A. J.: US20036524466 (2003). [39 Christ, F. R.: U.S. Pat. No. 5,843,186 (1998). [4] Holt K B, Bard A J. Interaction of silver (I) ions with the respiratory chain of Escherichia coli: an electrochemical and scanning electrochemical microscopy study of the antimicrobial mechanism of micromolar Ag+. Biochem 2005; 44: 13214-23. [5] Darouiche R O. Anti-infective efficacy of silver-coated medical prostheses. Clin Infect Dis 1999; 29: 1371-77. [6] Milder, F. L.: U.S. Pat. No. 5,725,817 (1998). [7] Prosl, F. R., Polaschegg, H.-D., Estabrook, B. K., Sodemann, K.: US20036575945 (2003). [89 Jamiolkowski, D. D., Rothenburger, S. J., Spangler, Daniel J.: US20036514517 (2003). [99 Jernberg, G. R.: US20046726898 (2004). [10] Itzhak B. The Role of Bacterial Interference in Otitis, Sinusitis and Tonsillitis, Otolaryngology—Head and Neck Surgery 2005; 133: 139-46. [11] Darouiche, R. O., Hull, R.: US20046719991 (2004). [12] Saint S. Clinical and economic consequences of nosocomial catheter-related bacteriuria. Am J Infection Control 2000; 28: 68-75. [13] Öncü S, Sakarya S. Central venous catheter-related infections: an overview with special emphasis on diagnosis, prevention and management. Internet J. Anesthesiology 2003; 7 (N. 1). [14] Seder, E. V., Nelson, J. N.: WO02083031A3 (2002). [15] Polaschegg, H.-D.: US20046803363 (2004). [16] Ash, S. R.: US20056958049 (2005). [17] Stephen, R. L., Rossi, C., Eruzzi, S.: U.S. Pat. No. 5,401,239 (1995). [18] Crossley, K.: US20036551346 (2003). [19] Acikel, C., O. Oncul, E. Ulkur, I. Bayram, B. Celikoz, and S. Cavuslu. 2003. Comparison of silver sulfadiazine 1%, mupirocin 2%, and fusidic acid 2% for topical antibacterial effect in methicillin-resistant staphylococci-infected, full-skin thickness rat burn wounds. J. Burn Care Rehabil. 24:37-41. [20] Altoparlak, U., S. Erol, M. N. Akcay, F. Celebi, and A. Kadanali. 2004. The time-related changes of antimicrobial resistance patterns and predominant bacterial profiles of burn wounds and body flora of burned patients. Burns 30:660-664. [219 Atiyeh, B. S., R. Dham, M. Kadry, A. F. Abdallah, M. Al-Oteify, O. Fathi, and A. Samir. 2002. Benefit-cost analysis of moist exposed burn ointment. Burns 28:659-663. [229 Atiyeh, B. S., K. A. El-Musa, and R. Dham. 2003. Scar quality and physiologic barrier function restoration after moist and moist-exposed dressings of partial-thickness wounds. Dermatol. Surg. 29:14-20. [23] Atiyeh, B. S., J. Ioannovich, G. Magliacani, M. Masellis, M. Costagliola, R. Dham, and M. Al-Farhan. 2002. Efficacy of moist exposed burn ointment in the management of cutaneous wounds and ulcers: a multicenter pilot study. Ann. Plast. Surg. 48:226-227. [24] Baddley, J. W., and S. A. Moser. 2004. Emerging fungal resistance. Clin. Lab. Med. 24:721-735, vii. [25] Barret, J. P., P. I. Ramzy, J. P. Heggers, C. Villareal, D. N. Herndon, and M. H. Desai. 1999. Topical nystatin powder in severe burns: a new treatment for angioinvasive fungal infections refractory to other topical and systemic agents. Burns 25:505-508. [26] Boyce, S. T., A. P. Supp, V. B. Swope, and G. D. Warden. 1999. Topical sulfamylon reduces engraftment of cultured skin substitutes on athymic mice. J. Burn Care Rehabil. 20:33-36. [27] Brown, T. P., L. C. Cancio, A. T. McManus, and A. D. Mason, Jr. 2004. [28] Choban, P. S., and W. J. Marshall. 1987. Leukopenia secondary to silver sulfadiazine: frequency, characteristics and clinical consequences. Am. Surg. 53:515-517. [29] Coghlan, A. 1996. Peptides punch it out with superbugs. New Sci. 150 (June):20. [30] Cooper, R. A., and P. C. Molan. 1999. Honey in wound care. J. Wound Care 8:340. [31] Cooper, M. L., S. T. Boyce, J. F. Hansbrough, T. J. Foreman, and D. H. Frank. 1990. Cytotoxicity to cultured human keratinocytes of topical antimicrobial agents. J. Surg. Res. 48:190-195. [32] Dunn, K., and V. Edwards-Jones. 2004. The role of Acticoat with nanocrystalline silver in the management of burns. Burns 30 (Suppl. 1):S1-S9. [33] Elliott, T. S., and P. A. Lambert. 1999. Antibacterial resistance in the intensive care unit: mechanisms and management. Br. Med. Bull. 55:259-276. [34] Embil, J. M., J. A. McLeod, A. M. Al-Barrak, G. M. Thompson, F. Y. Aoki, E. J. Witwicki, M. F. Stranc, A. M. Kabani, D. R. Nicoll, and L. E. Nicolle. 2001. An outbreak of methicillin resistant Staphylococcus aureus on a burn unit: potential role of contaminated hydrotherapy equipment. Burns 27: 681-688. [35] Falcone, A. E., and J. A. Spadaro. 1986. Inhibitory effects of electrically activated silver material on cutaneous wound bacteria. Plast. Reconstr. Surg. 77:455-459. [36] Falcone, P. A., H. N. Harrison, G. O. Sowemimo, and G. P. Reading. 1980. Mafenide acetate concentrations and bacteriostasis in experimental burn wounds treated with a three-layered laminated mafenide-saline dressing. Ann. Plast. Surg. 5:266-269. [37] Fuller, F. W., and P. E. Engler. 1988. Leukopenia in non-septic burn patients receiving topical 1% silver sulfadiazine cream therapy: a survey. J. Burn Care Rehabil. 9:606-609. [38] Garner, J. P., and P. S. Heppell. 2005. The use of Flammacerium in British burns units. Burns 31:379-382. [39] Garner, J. P., and P. S. Heppell. 2005. Cerium nitrate in the management of burns. Burns 31:539-547. [40] Greener, M. 1998. The fifty year war against infection. Pharm. Times 1998(June):34-37. [41] Heggers, J., R. E. Goodheart, J. Washington, L. McCoy, E. Carino, T. Dang, P. Edgar, C. Maness, and D. Chinkes. 2005. Therapeutic efficacy of three silver dressings in an infected animal model. J. Burn Care Rehabil. 26:53-56. [42] Heggers, J. P., H. Hawkins, P. Edgar, C. Villarreal, and D. N. Herndon. 2002. Treatment of infections in burns, p. 120-169. In D. N. Herndon (ed.) Total burn care. Saunders, London, England. [43] Heggers, J. P., M. C. Robson, D. N. Herndon, and M. H. Desai. 1989. The efficacy of nystatin combined with topical microbial agents in the treatment of burn wound sepsis. J. Burn Care Rehabil. 10:508-511. [44] Herruzo-Cabrera, R., V. Garcia-Torres, J. Rey-Calero, and M. J. Vizcaino-Alcaide. 1992. Evaluation of the penetration strength, bactericidal efficacy and spectrum of action of several antimicrobial creams against isolated microorganisms in a burn centre. Burns 18:39-44. [45] Lansdown, A. B. 2002. Silver. 2. Toxicity in mammals and how its products aid wound repair. J. Wound Care 11:173-177. [46] Lansdown, A. B. 2002. Silver. 1. Its antibacterial properties and mechanism of action. J. Wound Care 11:125-130. [47] Lansdown, A. B., A. Williams, S. Chandler, and S. Benfield. 2005. Silver absorption and antibacterial efficacy of silver dressings. J. Wound Care 14:155 160. [489 MacMillan, B. G., E. O. Hill, and W. A. Altemeier. 1967. Use of topical silver nitrate, mafenide, and gentamicin in the burn patient. A comparative study. Arch. Surg. 95:472-481. [49] Meier, P. A., C. D. Carter, S. E. Wallace, R. J. Hollis, M. A. Pfaller, and L. A. Herwaldt. 1996. A prolonged outbreak of methicillin-resistant Staphylococcus aureus in the burn unit of a tertiary medical center. Infect. Control Hosp. Epidemiol. 17:798-802. [509 Molan, P. C. 1999. The role of honey in the management of wounds. J. Wound Care 8:415-418. [51] Monafo, W. W., and M. A. West. 1990. Current treatment recommendations for topical burn therapy. Drugs 40:364-373. [52] Mousa, H. A., and S. M. al-Bader. 2001. Yeast infection of burns. Mycoses 44:147-149. [53] Murphy, K. D., J. O. Lee, and D. N. Herndon. 2003. Current pharmacotherapy for the treatment of severe burns. Expert Opin. Pharmacother. 4:369-384. [54] Namias, N., L. Samiian, D. Nino, E. Shirazi, K. O'Neill, D. H. Kett, E. Ginzburg, M. G. McKenney, D. Sleeman, and S. M. Cohn. 2000. Incidence and susceptibility of pathogenic bacteria vary between intensive care units within a single hospital: implications for empiric antibiotic strategies. J. Trauma 49:638-645. [55] Palmieri, T. L., and D. G. Greenhalgh. 2002. Topical treatment of pediatric patients with burns: a practical guide. Am. J. Clin. Dermatol. 3:529-534. [56] Pirnay, J. P., D. De Vos, C. Cochez, F. Bilocq, J. Pirson, M. Struelens, L. Duinslaeger, P. Cornelis, M. Zizi, and A. Vanderkelen. 2003. Molecular epidemiology of Pseudomonas aeruginosa colonization in a burn unit: persistence of a multidrug-resistant clone and a silver-sulfadiazine-resistant clone. J. Clin. Microbiol. 41:1192-1202. [57] Rode, H., D. Hanslo, P. M. De Wet, A. J. Millar, and S. Cyeses. 1989. Efficacy of mupirocin in methicillin-resistant Staphylococcus aureus burn wound infection. Antimicrob. Agents Chemother. 33:1358-1361. [58] Rodgers, G. L., J. E. Mortensen, M. C. Fisher, and S. S. Long. 1997. In vitro susceptibility testing of topical antimicrobial agents used in pediatric burn patients: comparison of two methods. J. Burn Care Rehabil. 18:406-410. [59] Stefanides, M. M., Sr., C. E. Copeland, S. D. Kominos, and R. B. Yee. 1976. In vitro penetration of topical antiseptics through eschar of burn patients. Ann. Surg. 183:358-364. [60] Strock, L. L., M. M. Lee, R. L. Rutan, M. H. Desai, M. C. Robson, D. N. Herndon, and J. P. Heggers. 1990. Topical Bactroban (mupirocin): efficacy in treating burn wounds infected with methicillin-resistant staphylococci. J. Burn Care Rehabil. 11:454-459. [61] Tredget, E. E., H. A. Shankowsky, A. Groeneveld, and R. Burrell. 1998. A matched-pair, randomized study evaluating the efficacy and safety of Acticoat silver-coated dressing for the treatment of burn wounds. J. Burn Care Rehabil. 19:531-537. [62] Wright, J. B., K. Lam, D. Hansen, and R. E. Burrell. 1999. Efficacy of topical silver against fungal burn wound pathogens. Am. J. Infect Control 27:344-350. [63] Yin, H. Q., R. Langford, and R. E. Burrell. 1999. Comparative evaluation of the antimicrobial activity of ACTICOAT antimicrobial barrier dressing. J. Burn Care Rehabil. 20:195-200.

Hence, medical devices, implants a wound dressing which comprises means for killing living target cells, or otherwise disrupting vital intracellular processes and/or intercellular interactions of said cells upon contact, are still an unmet need.

### SUMMARY OF THE INVENTION

It is one object of the invention to disclose a medical device, especially a medical device selected from a group consisting inter alia of medical devices, implants wound dressings, comprising at least one insoluble proton sink or source (PSS). The medical device is provided useful for killing living target cells (LTCs), or otherwise disrupting vital intracellular processes and/or intercellular interactions of the LTC upon contact. The PSS comprising (i) proton source or sink providing a buffering capacity; and (ii) means providing proton conductivity and/or electrical potential; wherein the PSS is effectively disrupting the pH homeostsis and/or electrical balance within the confined volume of the LTC and/or disrupting vital intercellular interactions of the LTCs while efficiently preserving the pH of the LTCs' environment.

It is another object of the invention to disclose the medical device as defined above, wherein the proton conductivity is provided by water permeability and/or by wetting, especially wherein the wetting is provided by hydrophilic additives.

It is another object of the invention to disclose the medical device as defined above, wherein the proton conductivity or wetting is provided by inherently proton conductive materials (IPCMs) and/or inherently hydrophilic polymers (IHPs), selected from a group consisting of sulfonated tetrafluortheylene copolymers; sulfonated materials selected from a group consisting of silica, polythion-ether sulfone (SPTES), styrene-ethylene-butylene-styrene (S-SEBS), polyether-ether-ketone (PEEK), poly (arylene-ether-sulfone) (PSU), Polyvinylidene Fluoride (PVDF)-grafted styrene, polybenzimidazole (PBI) and polyphosphazene; proton-exchange membrane made by casting a polystyrene sulfonate (PSSnate) solution with suspended micron-sized particles of cross-linked PSSnate ion exchange resin; commercially available Nafion™ and derivatives thereof.

It is another object of the invention to disclose the medical device as defined above, wherein the device comprising two or more, either two-dimensional (2D) or three-dimensional (3D) PSSs, each of which of the PSSs consisting of materials containing highly dissociating cationic and/or anionic groups (HDCAs) spatially organized in a manner which efficiently minimizes the change of the pH of the LTC's environment; each of the HDCAs is optionally spatially organized in specific either 2D, topologically folded 2D surfaces, or 3D manner efficiently which minimizes the change of the pH of the LTC's environment; further optionally, at least a portion of the spatially organized HDCAs are either 2D or 3D positioned in a manner selected from a group consisting of (i) interlacing; (ii) overlapping; (iii) conjugating; (iv) either homogeneously or heterogeneously mixing and (iv) tailing the same

It is another object of the invention to disclose the medical device as defined above, wherein the PSS is effectively disrupting the pH homeostasis within a confined volume while efficiently preserving the entirety of the LTC's environment; and further wherein the environment's entirety is characterized by parameters selected from a group consisting of the environment functionality, chemistry; soluble's concentration, possibly other then proton or hydroxyl concentration; biological related parameters; ecological related parameters; physical parameters, especially particles size distribution, rehology and consistency; safety parameters, especially toxicity, otherwise LD50 or ICT50 affecting parameters; olphactory or organoleptic parameters (e.g., color, taste, smell, texture, conceptual appearance etc); or any combination of the same.

It is acknowledged in this respect to underline that the term HDCAs refers, according to one specific embodiment of the invention, and in a non-limiting manner, to ion-exchangers, e.g., water immiscible ionic hydrophobic materials.

It is another object of the invention to disclose the medical device as defined above, wherein the device is provided useful for disrupting vital intracellular processes and/or intercellular interactions of the LTC, while both (i) effectively preserving the pH of the LTC's environment, and (ii) minimally affecting the entirety of the LTC's environment such that a leaching from the PSS of either ionized or electrically neutral atoms, molecules or particles (AMP) to the LTC's environment is minimized.

It is well in the scope of the invention wherein the aforesaid leaching minimized such that the concentration of leached ionized or neutral atoms is less than 1 ppm. Alternatively, the aforesaid leaching is minimized such that the concentration of leached ionized or neutral atoms is less than 50 ppb. Alternatively, the aforesaid leaching is minimized such that the concentration of leached ionized or neutral atoms is less than 50 ppb and more than 10 ppb. Alternatively, the aforesaid leaching is minimized such that the concentration of leached ionized or neutral atoms is less than 10 but more than 0.5 ppb. Alternatively, the aforesaid leaching is minimized such that the concentration of leached ionized or neutral atoms is less than 0.5 ppb.

It is another object of the invention to disclose the medical device as defined above, wherein the device is provided useful for disrupting vital intracellular processes and/or intercellular interactions of the LTC, while less disrupting pH homeostasis and/or electrical balance within at least one second confined volume (e.g., non-target cells, NTC).

It is another object of the invention to disclose the medical device as defined above, wherein the differentiation between the LTC and NTC is obtained by one or more of the following means (i) providing differential ion capacity; (ii) providing differential pH values; and, (iii) optimizing PSS to target cell size ratio; (iv) providing a differential spatial, either 2D, topologically 2D folded surfaces, or 3D configuration of the PSS; (v) providing a critical number of PSS' particles (or applicable surface) with a defined capacity per a given volume; and (vi) providing size exclusion means.

It is another object of the invention to disclose the medical device as defined above, comprising at least one insoluble non-leaching PSS as defined in any of the above, wherein the PSS, located on the internal and/or external surface of the medical device, is provided useful, upon contact, for disrupting pH homeostasis and/or electrical balance within at least a portion of an LTC while effectively preserving pH & functionality of the surface.

It is another object of the invention to disclose the medical device as defined above, wherein the device is provided useful for target cell's killing, the method is having at least one external proton-permeable surface with a given functionality (e.g., electrical current conductivity, affinity, selectivity etc), the surface is at least partially composed of, or topically and/or underneath layered with a PSS, such that disruption of vital intracellular processes and/or intercellular interactions of the LTC is provided, while the LTC's environment's pH & the functionality is effectively preserved.

It is another object of the invention to disclose the medical device as defined above, the device further comprising a surface with a given functionality, and one or more external proton-permeable layers, each of which of the layers is disposed on at least a portion of the surface; wherein the layer is at least partially composed of or layered with a PSS such that vital intracellular processes and/or intercellular interactions of the LTC are disrupted, while the LTC's environment's pH & the functionality is effectively preserved.

It is another object of the invention to disclose the medical device as defined above, the device further comprising (i) at least one PSS; and (ii) one or more preventive barriers, providing the PSS with a sustained long activity; preferably wherein at least one barrier is a polymeric preventive barrier adapted to avoid heavy ion diffusion; further preferably wherein the polymer is an ionomeric barrier, and particularly a commercially available Nafion™.

It is acknowledged in this respect that the presence or incorporation of barriers that can selectively allow transport of protons and hydroxyls but not of other competing ions to and/or from the solid ion exchanger (SIEX) surface eliminates or substantially reduces the ion-exchange saturation by counter-ions, resulting in sustained and long acting cell killing activity of the materials and compositions of the current invention.

It is in the scope of the invention, wherein the proton and/or hydroxyl-exchange between the cell and strong acids and/or strong basic materials and compositions may lead to disruption of the cell pH-homeostasis and consequently to cell death. The proton conductivity property, the volume buffer capacity and the bulk activity are pivotal and crucial to the present invention.

It is further in the scope of the invention, wherein the pH derived cytotoxicity can be modulated by impregnation and coating of acidic and basic ion exchange materials with polymeric and/or ionomeric barrier materials.

It is another object of the invention to disclose the medical device as defined above, the device further adapted to avoid development of LTC's resistance and selection over resistant mutations.

It is another object of the invention to disclose the medical device as defined above, wherein the device further comprising designed as a continuous barrier the barrier is selected from a group consisting of either 2D or 3D membranes, filters, meshes, nets, sheet-like members or a combination thereof.

It is another object of the invention to disclose the medical device as defined above, wherein the device further designed as an insert, comprising at least one PSS, the insert is provided with dimensions adapted to ensure either (i) reversibly mounting or (ii) permanent accommodation of the insert within a predetermined article of manufacture.

It is another object of the invention to disclose the medical device as defined above, wherein the device further characterized by at least one of the following (i) regeneratable proton source or sink; (ii) regeneratable buffering capacity; and (iii) regeneratable proton conductivity.

It is another object of the invention to disclose a method for killing living target cells (LTCs), or otherwise disrupting vital intracellular processes and/or intercellular interactions of the LTC being in contact with a medical device; the method comprising steps of: providing the medical device with at least one PSS having (i) proton source or sink providing a buffering capacity; and (ii) means providing proton conductivity and/or electrical potential; contacting the LTCs with the PSS; and, by means of the PSS, effectively disrupting the pH homeostasis and/or electrical balance within the LTC while efficiently preserving the pH of the LTC's environment.

It is another object of the invention to disclose a method as defined above, wherein the step (a) further comprising a step of providing the PSS with water permeability and/or wetting characteristics, in particular wherein the proton conductivity and wetting is at least partially obtained by providing the PSS with hydrophilic additives.

It is another object of the invention to disclose a method as defined above, wherein the method further comprising a step of providing the PSS with inherently proton conductive materials (IPCMs) and/or inherently hydrophilic polymers (IHPs), especially by selecting the IPCMs and/or IHPs from a group consisting of sulfonated tetrafluoroethylene copolymers; commercially available Nafion™ and derivatives thereof.

It is another object of the invention to disclose a method as defined above, the method further comprising steps of providing the medical device with two or more, either two-dimensional (2D), topologically folded 2D surfaces or three-dimensional (3D) PSSs, each of which of the PSSs consisting of materials containing highly dissociating cationic and/or anionic groups (HDCAs); and, spatially organizing the HDCAs in a manner which minimizes the change of the pH of the LTC's environment.

It is another object of the invention to disclose a method as defined above, the method further comprising a step of spatially organizing each of the HDCAs in a specific, either 2D or 3D manner, such that the change of the pH of the LTC's environment is minimized .

It is another object of the invention to disclose a method as defined above, wherein the step of organizing is provided by a manner selected for a group consisting of (0 interlacing the HDCAs; (ii) overlapping the HDCAs; (iii) conjugating the HDCAs; (iv) either homogeneously or heterogeneously mixing the HDCAs; and (v) tiling of the same

It is another object of the invention to disclose a method as defined above, the method further comprising a step of disrupting pH homeostasis and/or electrical potential within at least a portion of an LTC by a PSS, while both (i) effectively preserving the pH of the LTC's environment; and (ii) minimally affecting the entirety of the LTC's environment; the method is especially provided by minimizing the leaching of either ionized or electrically neutral atoms, molecules or particles (AMP) from the PSS to the LTC's environment.

It is another object of the invention to disclose a method as defined above, the method further comprising steps of preferentially disrupting pH homeostasis and/or electrical balance within at least one first confined volume (e.g., target living cells, LTC), while less disrupting pH homeostasis within at least one second confined volume (e.g., non-target cells , NTC).)

It is another object of the invention to disclose a method as defined above, wherein the differentiation between the LTC and NTC is obtained by one or more of the following steps: (i) providing differential ion capacity; (ii) providing differential pH value; (iii) optimizing the PSS to LTC size ratio; and, (iv) designing a differential spatial configuration of the PSS boundaries on top of the PSS bulk; and (v) providing a critical number of PSS' particles (or applicable surface) with a defined capacity per a given volume; and (vi) providing size exclusion means, e.g., mesh, grids etc.)

It is another object of the invention to disclose a method for the production of a medical device, comprising steps of providing a medical device as defined in any of the above; comprising steps of locating the PSS on top or underneath the surface of the medical device; and upon contacting the PSS with a LTC, disrupting the pH homeostasis and/or electrical balance within at least a portion of the LTC while effectively preserving pH & functionality of the surface.

It is another object of the invention to disclose a method as defined above, the method further comprising steps of: providing the medical device with at least one external proton-permeable surface with a given functionality; and, providing at least a portion of the surface with at least one PSS, and/or layering at least one PSS on top of underneath the surface; hence killing LTCs or otherwise disrupting vital intracellular processes and/or intercellular interactions of the LTC, while effectively preserving the LTC's environment's pH & functionality.

It is another object of the invention to disclose a method as defined above, the method further comprising steps of: providing the medical device with at least one external proton-permeable providing a surface with a given functionality; disposing one or more external proton-permeable layers topically and/or underneath at least a portion of the surface; the one or more layers are at least partially composed of or layered with at least one PSS; and, killing LTCs, or otherwise disrupting vital intracellular processes and/or intercellular interactions of the LTC, while effectively preserving the LTC's environment's pH & functionality.)

It is another object of the invention to disclose a method as defined above, the method further comprising steps of providing the medical device with at least one PSS; and, providing the PSS with at least one preventive barrier such that a sustained long acting is obtained.)

It is another object of the invention to disclose a method as defined above, wherein the step of providing the barrier is obtained by utilizing a polymeric preventive barrier adapted to avoid heavy ion diffusion; preferably by providing the polymer as an ionomeric barrier, and particularly by utilizing a commercially available Nafion™ product.)

It is hence in the scope of the invention wherein one or more of the following materials are provided: encapsulated strong acidic and strong basic buffers in solid or semi-solid envelopes, solid ion-exchangers (SIEx), ionomers, coated-SIEx, high-cross-linked small-pores SIEx, Filled-pores SIEx, matrix-embedded SIEx, ionomeric particles embedded in matrices, mixture of anionic (acidic) and cationic (basic) SIEx etc.

It is another object of the invention to disclose the PSS as defined in any of the above, wherein the PSS are naturally occurring organic acids compositions containing a variety of carbocsylic and/or sulfonic acid groups of the family, abietic acid (C₂₀H₃₀O₂) such as colophony/rosin, pine resin and alike, acidic and basic terpenes.

It is another object of the invention to disclose a method for inducing apoptosis in at least a portion of LTCs population in a medical device. The method comprising steps of: obtaining at least one medical device as defined in any of the above; contacting the PSS with an LTC; and, effectively disrupting the pH homeostasis and/or electrical balance within the LTC such that the LTC's apoptosis is obtained, while efficiently preserving the pH of the LTC's environment and patient's safety.

It is another object of the invention to disclose a method for avoiding development of LTC's resistance and selecting over resistant mutations. The method comprising steps of: obtaining at least one medical device as defined in any of the above; contacting the PSS with an LTC; and, effectively disrupting the pH homeostasis and/or electrical balance within the LTC such that development of LTC's resistance and selecting over resistant mutations is avoided, while efficiently preserving the pH of the LTC's environment and patient's safety.

It is another object of the invention to disclose a method of regenerating the biocidic properties of a medical device as defined in any of the above; comprising at least one step selected from a group consisting of (i) regenerating the PSS; (ii) regenerating its buffering capacity; and (iii) regenerating its proton conductivity.

It is another object of the invention to disclose a method of treating a patient, either human or animal. The method comprising steps of administrating to a patient, via a medical device, implant or wound dressing, an effective measure of PSSs as defined in any of the above, in a manner the PSSs contacts at least one LTC; and disrupting vital intracellular processes and/or intercellular interactions of the LTC, while effectively preserving the pH of the LTC's environment

It is in the scope of the invention wherein an effective dose of the PSS is administrated e.g., orally, rectally, topically or intravenously, as a particulate matter, provided as is or by a pharmaceutically accepted carrier. The administration may be provided in a sustained release form the medical devices, implants or wound dressings of the present invention. or by any other suitable means. Hence, the PSS is soaked, doped, immersed, contained, immobilized or otherwise bonded to the either inner or outer surface of the medical devices, implants or wound dressings, and may comprise or contained with effective measure of additives.

### BRIEF DESCRIPTION OF THE DRAWINGS

In order to understand the invention and to see how it may be implemented in practice, a plurality of preferred embodiments will now be described, by way of non-limiting example only, with reference to the accompanying drawing, in which

FIG. 1 showing a standard MIC test with double-diluted Poly-(4-styrenesulfonic acid) on TSA plates inoculated with starter culture of S. caseolitycus;

FIG. 2 presenting photographs of standard MIC test with double-diluted Poly-(4-styrenesulfonic acid) on TSA plates inoculated with starter culture of S. caseolitycus;

FIG. 3 showing a standard MIC test with double-diluted PSSA on TSA plates inoculated with starter culture of S. caseolitycus

FIG. 4 showing the antimicrobial activity of Amberlite 120 and Amberlite (H⁺-form) applied to standard, commercially available plaster against S. caseolitycus;

FIG. 5 showing the antimicrobial activity of Amberlite 120+Ascorbic acid applied to standard, commercially available plaster against S. caseolitycus;

FIG. 6 presenting the antimicrobial activity of Poly-(4-styrenesulfonic acid) (PSSA) applied to standard, commercially available plaster against S. caseolitycus.

FIG. 7 presenting P. acnes growing on CDC blood agar and incubated in 37° C. under anaerobic condition;

FIG. 8 showing antimicrobial activity of Amberlite 120, Amberlite (H⁺ and OH⁻ forms), Amberlite+Ascorbic acid and PSSA against P. acnes;

FIG. 9 showing treatment of Acne lesions on a human volunteer with commercially available plasters amended with PSSA; and,

FIG. 10, presenting a treatment of Acne lesions on a human volunteer with commercially available plasters amended

### DESCRIPTION OF THE PREFERRED EMBODIMENTS

The following specification taken in conjunction with the drawings sets forth the preferred embodiments of the present invention. The embodiments of the invention disclosed herein are the best modes contemplated by the inventors for carrying out their invention in a commercial environment, although it should be understood that various modifications can be accomplished within the parameters of the present invention.

The term ‘contact’ refers hereinafter to any direct or indirect contact of a PSS with a confined volume (living target cell or virus—LTC), wherein the PSS and LTC are located adjacently, e.g., wherein the PSS approaches either the internal or external portions of the LTC; further wherein the PSS and the LTC are within a proximity which enables (i) an effective disruption of the pH homeostasis and/or electrical balance, or (ii) otherwise disrupting vital intracellular processes and/or intercellular interactions of the LTC.

The terms ‘effectively’ and ‘effectively’ refer hereinafter to an effectiveness of over 10%, additionally or alternatively, the term refers to an effectiveness of over 50%; additionally or alternatively, the term refers to an effectiveness of over 80%. It is in the scope of the invention, wherein for purposes of killing LTCs, the term refers to killing of more than 50% of the LTC population in a predetermined time, e.g., 10 min.

The term ‘additives’ refers hereinafter to one or more members of a group consisting of biocides e.g., organic biocides such as tea tree oil, rosin, abietic acid, terpens, rosemary oil etc, and inorganic biocides, such as zinc oxides, copper and mercury, silver salts etc, markers, biomarkers, dyes, pigments, radio-labeled materials, glues, adhesives, lubricants, medicaments, sustained release drugs, nutrients, peptides, amino acids, polysaccharides, enzymes, hormones, chelators, multivalent ions, emulsifying or de-emulsifying agents, binders, fillers, thickfiers, factors, co-factors, enzymatic-inhibitors, organoleptic agents, carrying means, such as liposomes, multilayered vesicles or other vesicles, magnetic or paramagnetic materials, ferromagnetic and non-ferromagnetic materials, biocompatibility-enhancing materials and/or biodegradating materials, such as polylactic acids and polyglutamine acids, anticorrosive pigments, anti-fouling pigments, UV absorbers, UV enhancers, blood coagulators, inhibitors of blood coagulation, e.g., heparin and the like, or any combination thereof.

The term ‘particulate matter’ refers hereinafter to one or more members of a group consisting of nano-powders, micrometer-scale powders, fine powders, free-flowing powders, dusts, aggregates, particles having an average diameter ranging from about 1 nm to about 1000 nm, or from about 1 mm to about 25 mm.

The term ‘about’ refers hereinafter to ±20% of the defined measure.

The term ‘medical device’ refers hereinafter in a non-limiting manner to items such as catheters, stents, endotracheal tubes, hypotubes, filters such as those for embolic protection, surgical instruments and the like. Any device that is typically coated in the medical arts and whose surface is capable of containing at least one PSS can be used in the present invention. It is further in the scope of the invention, wherein the term refers to any material, natural or artificial that is inserted into a mammal. Particular medical devices especially suited for application of the antimicrobial combinations of this invention include, but are not limited to, peripherally insertable central venous catheters, dialysis catheters, long term tunneled central venous catheters, long term non-tunneled central venous catheters, peripheral venous catheters, short-term central venous catheters, arterial catheters, pulmonary artery Swan-Ganz catheters, urinary catheters, artificial urinary sphincters, long term urinary devices, urinary dilators, urinary stents, other urinary devices, tissue bonding urinary devices, penile prostheses, vascular grafts, vascular catheter ports, vascular dilators, extravascular dilators, vascular stents, extravascular stents, wound drain tubes, hydrocephalus shunts, ventricular catheters, peritoneal catheters, pacemaker systems, small or temporary joint replacements, heart valves, cardiac assist devices and the like and bone prosthesis, joint prosthesis and dental prosthesis.

The term ‘implant’ refers hereinafter in a non-limiting manner to an artificial device embedded or transplanted into the human or animal body for medical purposes.

The term ‘wound dressing’ refers hereinafter in a non-limiting manner to any pharmaceutically acceptable wound covering, such as: a) films, including those of a semipermeable or a semi-occlusive nature such as polyurethane copolymers, acrylamides, acrylates, paraffin, polysaccharides, cellophane and lanolin. b) hydrocolloids including carboxymethylcellulose, protein constituents of gelatin, pectin, and complex polysaccharides including Acacia gum, guar gum and karaya. These materials may be utilized in the form of a flexible foam or, in the alternative, formulated in polyurethane or, in a further alternative, formulated as an adhesive mass such as polyisobutylene. c) hydrogels such as agar, starch or propylene glycol; which typically contain about 80% to about 90% water and are conventionally formulated as sheets, powders, pastes and gels in conjunction with cross-linked polymers such as polyethylene oxide, polyvinyl pyrollidone, acrylamide, propylene glycol. d) foams such as polysaccharide analogs which consist of a hydrophilic open-celled contact surface and hydrophobic closed-cell polyurethane e) impregnates including pine mesh gauze, paraffin and lanolin-coated gauze, polyethylene glycol-coated gauze, knitted viscose, rayon, and polyester. f) cellulose-like polysaccharides such as alginates, including calcium alginate, which may be formulated as non-woven composites of fibers or spun into woven composites.

The present invention relates to materials, compositions and methods for prevention of bacterial development in medical devices, wound dressing, implants and medical equipment by coating and/or incorporating the materials and compositions of the current invention in such a way that they will be capable of inhibiting bacterial proliferation and biofilm formation.

The biocidic activity, e.g., antibacterial activity, is based on preferential proton and/or hydroxyl-exchange between the cell and strong acids and/or strong basic materials and compositions. The materials and compositions of the present invention exert their cell killing effect via a titration-like process in which the said cell (e.g. bacteria, yeast, fungi etc.) is coming into contact with strong acids and/or strong basic buffers and the like: encapsulated strong acidic and strong basic buffers in solid or semi-solid envelopes, solid ion-exchangers (SIEx), ionomers, coated-SIEx, high-cross-linked small-pores SIEx, Filled-pores SIEx, matrix-embedded SIEx, Ionomeric particles embedded in matrices, mixture of anionic (acidic) and cationic (basic) SIEx etc. This process leads to disruption of the cell pH-homeostasis and consequently to cell death. The proton conductivity property, the volume buffer capacity and the bulk activity are pivotal and crucial to the present invention. The presence or incorporation of barriers that can selectively allow transport of protons and hydroxyls but not of other competing ions to and/or from the SIEx surface eliminates or substantially reduces the ion-exchange saturation by counter-ions, resulting in sustained and long acting cell killing activity of the materials and compositions of the current invention.

The materials and compositions of the current invention include but not limited to the following: all materials and compositions disclosed in PCT application No. PCT/IL2006/001262. The above mentioned materials and compositions of PCT/IL2006/001262 modified in such a way that these compositions are ion-selective by, for example: coating them with a selective coating, or ion-selective membrane; coating or embedding in high-cross-linked size excluding polymers etc; Strong acidic and strong basic buffers encapsulated in solid or semi-solid envelopes; SIEx particles—coated and non-coated, alone or in a mixture, embedded in matrices so as to create a pH-modulated polymer; SIEx particles—coated and non-coated, embedded in porous ceramic or glass water permeable matrices; Polymers which are alternately tiled with areas of high and low pH to create a mosaic-like polymer with an extended cell-killing spectrum; In addition to ionomers disclosed in the above mentioned PCT No. PCT/IL2006/001262, other ionomers can be used in the current invention as cell-killing materials and compositions. These may include, but certainly not limited to, for example: sulfonated silica, sulfonated polythion-ether sulfone (SPTES), sulfonated styrene-ethylene-butylene-styrene (S-SEBS), polyether-ether-ketone (PEEK), poly (arylene-ether-sulfone) (PSU), Polyvinylidene Fluoride (PVDF)-grafted styrene, polybenzimidazole (PBI) and polyphosphazene, proton-exchange membrane made by casting a polystyrene sulfonate (PSS) solution with suspended micron-sized particles of cross-linked PSS ion exchange resin.

All of the above mentioned materials and compositions of the current invention can be cast, molded or extruded and be used as particles in suspension, spray, cream, as membranes, coated films, fibers or fabrics, particles linked to or absorbed on fibers or fabrics, incorporated in filters etc.

It is in the scope of the invention, wherein a medical device, selected e.g., from a group consisting of medical devices, implants, wound dressings, comprises an insoluble PSS in the form of a polymer, ceramic, gel, resin or metal oxide is disclosed. The PSS is carrying strongly acidic or strongly basic functional groups (or both) adjusted to a pH of about <4.5 or about >8.0. It is in the scope of the invention, wherein the insoluble PSS is a solid buffer.

It is also in the scope of the invention wherein material's composition is provided such that the groups are accessible to water whether they are on the surface or in the interior of the PSS. Contacting a living cell (e.g., bacteria, fungi, animal or plant cell) with the PSS kills the cell in a time period and with an effectiveness depending on the pH of the PSS, the mass of PSS contacting the cell, the specific functional group(s) carried by the PSS, and the cell type. The cell is killed by a titration process where the PSS causes a pH change within the cell. The cell is often effectively killed before membrane disruption or cell lysis occurs. The PSS kills cells without directly contacting the cells if contact is made through a coating or membrane which is permeable to water, H+ and OH— ions, but not other ions or molecules. Such a coating also serves to prevent changing the pH of the PSS or of the solution surrounding the target cell by diffusion of counterions to the PSS's functional groups. It is acknowledged in thos respect that prior art discloses cell killing by strongly cationic (basic) molecules or polymers where killing probably occurs by membrane disruption and requires contact with the strongly cationic material or insertion of at least part of the material into the outer cell membrane.

It is also in the scope of the invention wherein an insoluble polymer, ceramic, gel, resin or metal oxide carrying strongly acid (e.g. sulfonic acid or phosphoric acid) or strongly basic (e.g. quaternary or tertiary amines) functional groups (or both) of a pH of about <4.5 or about >8.0 is disclosed. The functional groups throughout the PSS are accessible to water, with a volumetric buffering capacity of about 20 to about 100 mM H⁺/l/pH unit, which gives a neutral pH when placed in unbuffered water (e.g., about 5<pH>about 7.5) but which kills living cells upon contact.

It is also in the scope of the invention wherein the insoluble polymer, ceramic, gel, resin or metal oxide as defined above is coated with a barrier layer permeable to water, H⁺and OH⁻ ions, but not to larger ions or molecules, which kills living cells upon contact with the barrier layer.

It is also in the scope of the invention wherein the insoluble polymer, ceramic, gel, resin or metal oxide as defined above is provided useful for killing living cells by inducing a pH change in the cells upon contact.

It is also in the scope of the invention wherein the insoluble polymer, ceramic, gel, resin or metal oxide as defined above is provided useful for killing living cells without necessarily inserting any of its structure into or binding to the cell membrane.

It is also in the scope of the invention wherein the insoluble polymer, ceramic, gel, resin or metal oxide as defined above is provided useful for killing living cells without necessarily prior disruption of the cell membrane and lysis.

It is also in the scope of the invention wherein the insoluble polymer, ceramic, gel, resin or metal oxide as defined above is provided useful for causing a change of about <0.2 pH units of a physiological solution or body fluid surrounding a living cell while killing the living cell upon contact.

It is also in the scope of the invention wherein the insoluble polymer, ceramic, gel, resin or metal oxide as defined above is provided in the form of shapes, a coating, a film, sheets, beads, particles, microparticles or nanoparticles, fibers, threads, powders and a suspension of these particles.

The current invention is based on the modification of the surfaces of the medical device, tubes, catheters, implants etc. with a thin layer of the materials of the current invention to prevent bacterial development and biofilm formation on the surface of the medical device, whether outside or inside the body.

Those coatings can be produced by methods known in industry like spin coating, internal spray processing, Thermoplastic spraying, Evaporative deposition, coating with a varnish or thin layer resin etc. and can be deposited on surfaces of polymers, glass, metals, paper or any other material used in the medical device industry.

In all these coatings the active antibacterial materials will be incorporated in a polymer matrix suitable for attachment on the medical device material.

### Example 1

AntiBacterial Tests

Materials and Methods

Staphylococcus caseolyticus was grown in TSB medium to a concentration of 10⁸ cfu/ml. Poly-(4-styrenesulfonic acid) (PSSA; Aldrich) solution (18% wt/vol. in water) consisting of 70 kD particles had been serial-double-diluted from 1:1 up to 1:32. Standard MIC test was carried out by placing antibiotic disks soaked with double-diluted Poly-(4-styrenesulfonic acid) on TSA plates inoculated with starter culture of S. caseolitycus. Plates were incubated over night in 30° C.

Results

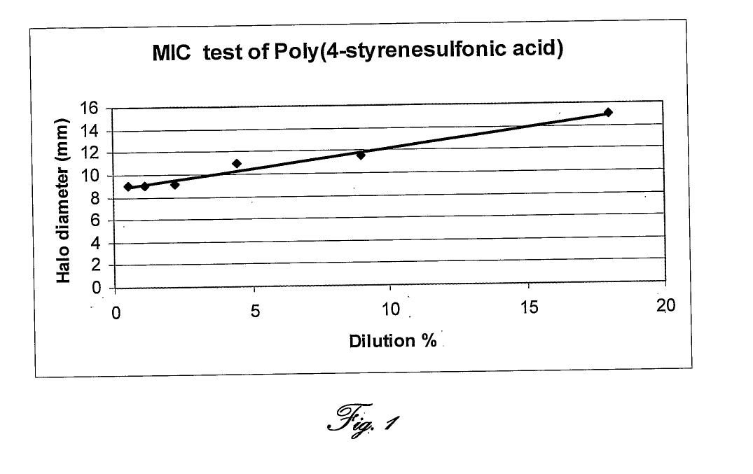

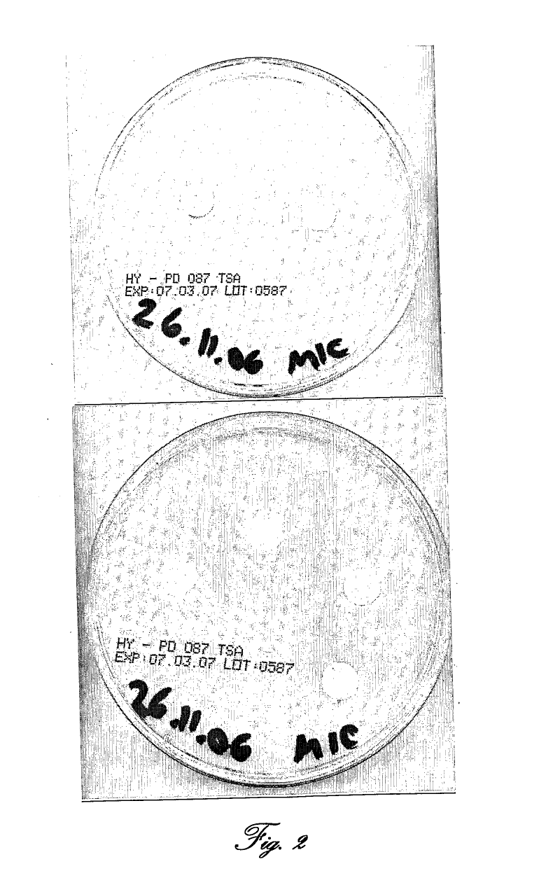

Reference is now made to FIG. 1, which shows a standard MIC test with double-diluted Poly-(4-styrenesulfonic acid) on TSA plates inoculated with starter culture of S. caseolitycus; and to FIG. 2, presenting photographs of standard MIC test with double-diluted Poly-(4-styrenesulfonic acid) on TSA plates inoculated with starter culture of S. caseolitycus.

Table 2 and FIGS. 1 and 2 shows an antimicrobial activity of PSSA against S. caseolyticus in concentrations as low as 2.25% of PSSA

TABLE 1
Standard MIC test with double-diluted Poly-(4-styrenesulfonic acid)
on TSA plates inoculated with starter culture of S. caseolitycus.

| | | Microbial growth Inhibition |
|---|---|---|
| Dilution | Concentration (%) | (Halo diameter in mm) |
| 1 | 18 | 15 |
| 1:2 | 9 | 11.5 |
| 1:4 | 4.5 | 11 |
| 1:8 | 2.25 | 9.2 |
| 1:16 | 1.125 | 9 |
| 1:32 | 0.5625 | 9 |

Similar results obtained with Nafion™ (commercially available product by Du Point) (perfluorinated resin solution 20 wt. % in mixture of lower aliphatic alcohols and water, contains 20% water; 527122 Aldrich) with Ascorbic Acid in Amberlite 120 and with PSSA (18%) in Biogel (Biorad).

### Example 2

Bacterial Resistance Test

Materials and methods—Staphylococcus caseolyticus was grown in TSB medium to a concentration of 108 cfu/ml. Poly-(4-styrenesulfonic acid) (Aldrich) solution (18% wt/vol. in water) consisting of 70 kD particles had been serial-double-diluted from 1:1 up to 1:32. Standard MIC test was carried out by placing antibiotic disks soaked with double-diluted Poly-(4-styrenesulfonic acid) (PSSA) on TSA plates inoculated with starter culture of S. caseolitycus. Plates were incubated over night in 30° C.

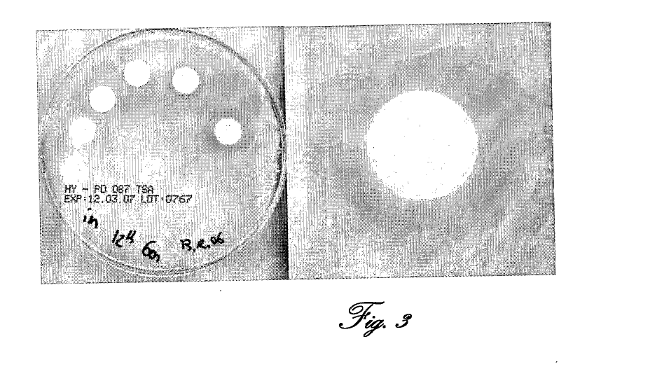

Samples of sensitive bacteria from inner and outer halo (cf. FIG. 3) had been taken with a needle stick and were seeded separately in TSB for a few hours and spread again on a new TSA plate for another MIC test with new Poly-(4-styrenesulfonic acid) disks. This test was performed again and again up to the 12^{th} bacterial generation.

Result Reference is now made to FIG. 3, showing a standard MIC test with double-diluted PSSA on TSA plates inoculated with starter culture of S. caseolitycus. Repeated MIC tests showed no change in bacterial behavior, and no resistance to Poly-(4-styrenesulfonic acid) could be observed. In a close up one can see inner and outer halo.

### Example 3

Antimicrobial Activity of Amberlite 120, Amberlite (H⁺-form), Amberlite+Ascorbic Acid and PSSA Applied to Standard Plaster (Band-Aid).

Materials and Methods

Amberlite 120, Amberlite (H⁺form), Amberlite+Ascorbic acid and PSSA were applied to standard, commercially available plaster (band-aid) and placed on TSA plates inoculated with starter culture of S. caseolitycus.Plates were incubated over night in 30° C. Antimicrobial activity was determined by the bacterial growth inhibition halos formed around application site.

Results

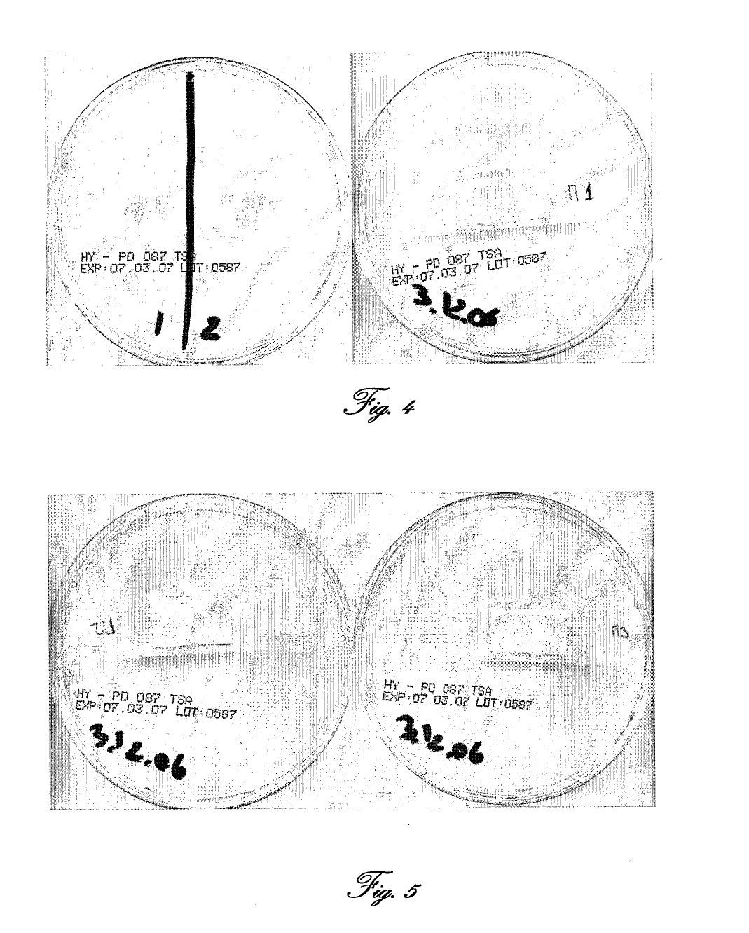

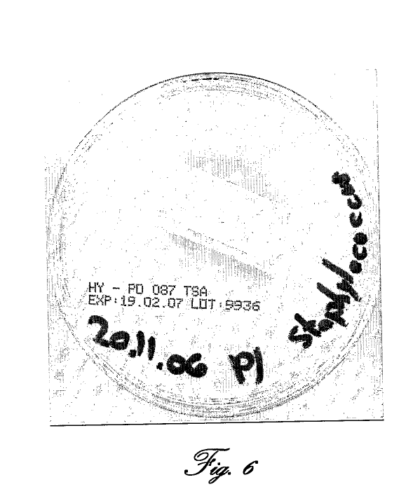

Reference is now made to FIG. 4, showing the antimicrobial activity of Amberlite 120 and Amberlite (H⁺form) applied to standard, commercially available plaster against S. caseolitycus, to FIG. 5, showing the antimicrobial activity of Amberlite 120 +Ascorbic acid applied to standard, commercially available plaster against S. caseolitycus; and to FIG. 6, presenting the antimicrobial activity of Poly-(4-styrenesulfonic acid) (PSSA) applied to standard, commercially available plaster against S. caseolitycus.

All material tested showed antimicrobial activity when applied to standard plasters (FIGS. 4, 5 and 6).

### Example 4

Antimicrobial Activity of Amberlite 120, Amberlite (H⁺-form), Amberlite+Ascorbic acid and PSSA Against Propionibacterium acnes

Materials and Methods

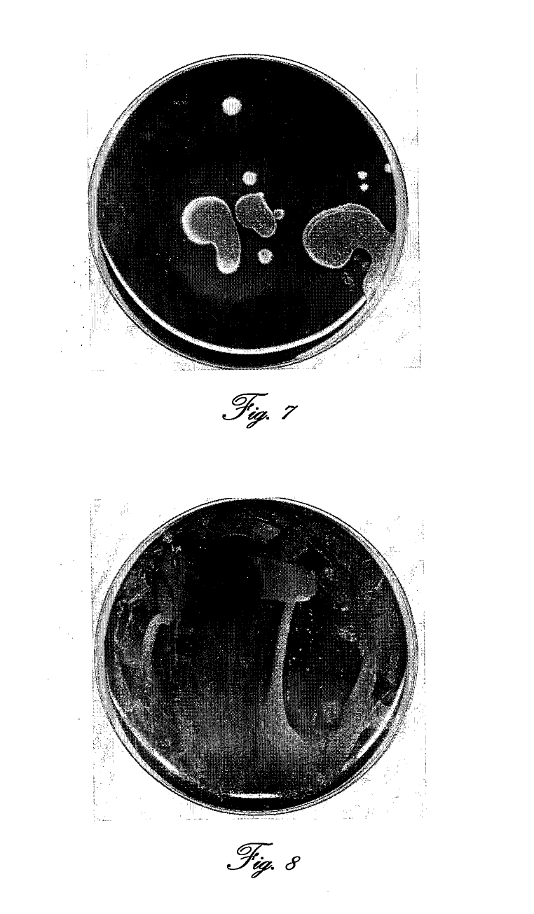

Reference is now made to FIG. 7, presenting P. acnes growing on CDC blood agar and incubated in 37° C. under anaerobic condition; and to FIG. 8. showing antimicrobial activity of Amberlite 120, Amberlite (H⁺and OH⁺ forms), Amberlite +Ascorbic acid and PSSA against P. acnes.

P. acnes was grown and maintained on CDC blood agar and incubated in 37° C. under anaerobic condition (cf. FIG. 7). The following materials were tested against P. acnes by applying them directly to a CDC blood agar plate inoculated with a lawn of the bacterium: Amberlite 120, Amberlite (H⁺ and OH⁻ forms), Amberlite+Ascorbic acid and PSSA. Antimicrobial activity was demonstrated by halos ob inhibition of bacterial growth around the application site (cf. FIG. 8).

### Example 5

Treatment of Acne Lesions on a Human Volunteer with Commercially Available Plasters Amended with PSSA

Materials and Methods

Commercially available plasters (Band-Aid) were amended with PSSA solution (18% wt/vol. in water) consisting of 70 kD particles. PSSA-amended plasters were air-dried in room temperature and placed on non-inflammatory Acne lesions on the forehead of a human volunteer. After three days, the plaster was removed and the size and severity of the lesions were observed.

Results

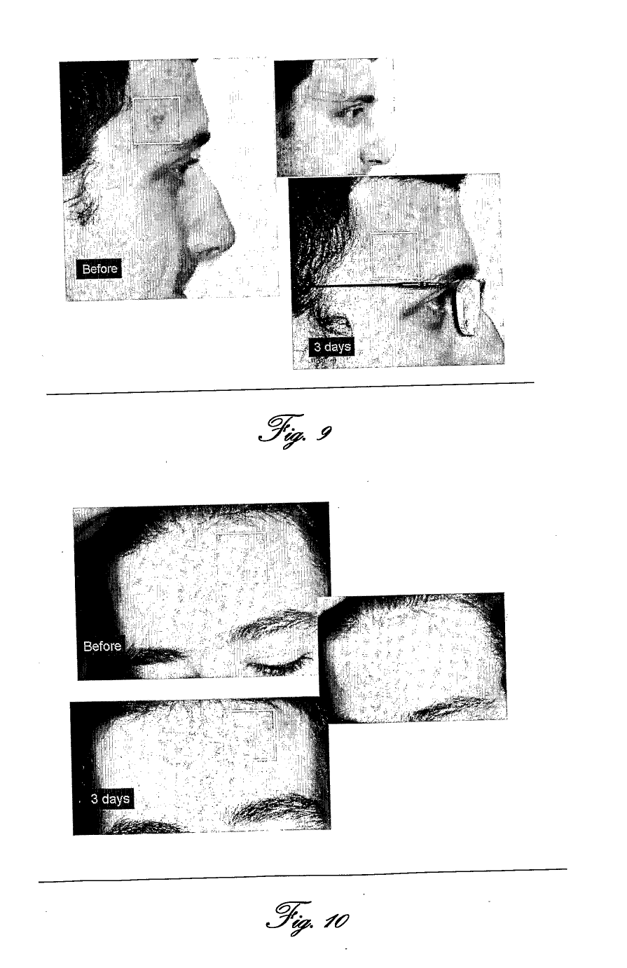

Reference is now made to FIG. 9, showing treatment of Acne lesions on a human volunteer with commercially available plasters amended with PSSA; and to FIG. 10, presenting a treatment of Acne lesions on a human volunteer with commercially available plasters amended.

Acne lesion size and severity were dramatically reduces after 3-days of treatment with PSSA-amended plaster. Lesions that were under direct exposure to the treatment were almost totally disappeared. Non-treated areas remained unchanged (FIGS. 9 and 10).

1-36. (canceled)

37. A medical device effective for killing cells, said medical device coated by at least one charged polymer, said at least one charged polymer characterized, when in contact with a body fluid, as:
a. carrying strongly acid and/or strongly basic functional groups;
b. having a pH of less than about 4.5 or greater than about 8.0;
c. capable of generating an electrical potential within the confined volume of said cell sufficient to disrupt effectively the pH homeostasis and/or electrical balance within said confined volume of said cell; and,
d. being in a form chosen from the group consisting of (i) H⁺ and (ii) OH⁻;
wherein said charged polymer is adapted to preserve the pH of said cell's environment.

38. The medical device of claim 37, wherein said medical device is chosen from the group consisting of catheters, stents, endotracheal tubes, hypotubes, filters, surgical instruments, peripherally insertable central venous catheters, dialysis catheters, long term tunneled central venous catheters, long term non-tunneled central venous catheters, peripheral venous catheters, short-term central venous catheters, arterial catheters, pulmonary artery Swan-Ganz catheters, urinary catheters, artificial urinary sphincters, long term urinary devices, urinary dilators, urinary stents, tissue bonding urinary devices, penile prostheses, vascular grafts, vascular catheter ports, vascular dilators, extravascular dilators, wound drain tubes, hydrocephalus shunts, ventricular catheters, peritoneal catheters, pacemaker systems, small or temporary joint replacements, heart valves, cardiac assist devices, bone prostheses, joint prostheses, and dental prostheses.

39. The medical device of claim 37, further characterized, when said groups are accessible to said body fluid, as having a buffering capacity of about 20 to about 100 mM H⁺/L/pH unit.

40. The medical device of claim 37, further characterized, when said groups are accessible to water, by at least one characteristic chosen from the group consisting of (a) sufficiently water-insoluble such that at least 99.9% remains undissolved at equilibrium; (b) sufficiently resistant to leaching such that the total concentration of material leached from said composition of matter into said body fluid does not exceed 1 ppm; (c) sufficiently inert such that at least one parameter of said body fluid chosen from the group consisting of (i) concentration of at least one predetermined water-soluble substance; (ii) particle size distribution; (iii) rheology; (iv) toxicity; (v) color; (vi) taste; (vii) smell; and (viii) texture remains unaffected according to preset conditions, said conditions adapted for and appropriate to said particular environment.

41. The medical device of claim 37, further comprising at least one polymer chosen from the group consisting of (a) polyvinyl alcohol; (b) polystyrene sulfonate; and (c) polypropylene polystyrene-divinylbenzene.

42. The medical device of claim 40, wherein at said at least one polymer contains at least one functional group chosen from the group consisting of 50₃H and H₂N(CH₃).

43. The medical device of claim 37, further comprising hydrophilic additives chosen from the group consisting of proton conductive materials (PCMs) and hydrophilic polymers (HPs); further wherein said PCMs and HPs are chosen from the group consisting of (a) sulfonated tetrafluoroethylene copolymers; (b) sulfonated materials chosen from the group consisting of silica, polythion-ether sulfone (SPTES), styrene-ethylene-butylene-styrene (S-SEBS), polyether-ether-ketone (PEEK), poly(arylene-ether-sulfone) (PSU), polyvinylidene fluoride (PVDF)-grafted styrene, polybenzimidazole (PBI), and polyphosphazene; and (c) proton-exchange membranes made by casting a polystyrene sulfonate (PSSnate) solution with suspended micron-sized particles of cross-linked PSSnate ion exchange resin.

44. The medical device of claim 37, comprising two or more charged polymers chosen from the group consisting of two-dimensional charged polymers and three-dimensional (3D) charged polymers, each of which of said charged polymers comprises materials containing cationic and/or anionic groups capable of dissociation and spatially organized in a manner adapted to preserve the pH of said body fluid according to preset conditions; said spatial organization chosen from the group consisting of (a) interlacing; (b) overlapping; (c) conjugating; (d) homogeneously mixing; (e) heterogeneously mixing; and (f) tiling.

45. The medical device of claim 37, further comprising a surface with a given functionality and at least one external proton-permeable layer, each of which of said at least one external proton-permeable layers is disposed on at least a portion of said surface.

46. The medical device of claim 37, adapted to avoid development of resistant mutations of said cells.

47. The medical device of claim 37, comprising at least one charged polymer and at least one barrier adapted to prevent heavy ion diffusion.

48. The medical device of claim 37, wherein said charged polymer is further characterized by at least one of the following:
a. capacity for absorbing or releasing protons capable of regeneration;
b. buffering capacity capable of regeneration; and
c. proton conductivity capable of regeneration.

49. A method for increasing the rate of death of living cells and/or decreasing the rate of reproduction of living cells within a body fluid, comprising the steps of:
a. providing a medical device comprising at least one charged polymer, said at least one charged polymer characterized, when in contact with said body fluid, as:
i. carrying strongly acid and/or strongly basic functional groups;
ii. having a pH of less than about 4.5 or greater than about 8.0;
iii. capable of generating an electrical potential within the confined volume of said cell sufficient to disrupt effectively the pH homeostasis and/or electrical balance within said confined volume of said cell; and,
iv. being in a form chosen from the group consisting of (i) H⁺ and (ii) OH⁻; and,
b. placing said medical device in contact with said body fluid.

50. The method of claim 49, wherein said step (a) further comprises the step of providing said charged polymer with predetermined water permeability, proton conductivity, and/or wetting characteristics, and further wherein said water permeability, proton conductivity, and/or wetting characteristics are provided by at least one substance selected from the group consisting of proton conductive materials (PCMB) and hydrophilic polymers (HPs).

51. The method of claim 50, wherein said step of providing said charged polymer with predetermined water permeability, proton conductivity, and/or wetting characteristics, and further wherein said water permeability, proton conductivity, and/or wetting characteristics are provided by at least one substance selected from the group consisting of proton conductive materials (PCMs) and hydrophilic polymers (HPs) further comprises a step of choosing said PCMs and HPs from the group consisting of (a) sulfonated tetrafluoroethylene copolymers; (b) sulfonated materials chosen from the group consisting of silica, polythion-ether sulfone (SPTES), styrene-ethylene-butylene-styrene (S-SEBS), polyether-ether-ketone (PEEK), poly(arylene-ether-sulfone) (PSU), polyvinylidene fluoride (PVDF)-grafted styrene, polybenzimidazole (PBI), and polyphosphazene; (c) proton-exchange membranes made by casting a polystyrene sulfonate (PSSnate) solution with suspended micron-sized particles of cross-linked PSSnate ion exchange resin; and derivatives thereof.

52. The method of claim 50, further comprising a step of providing at least one polymer chosen from the group consisting of (a) polyvinyl alcohol; (b) polystyrene sulfonate; and (c) polypropylene polystyrene-divinylbenzene.

53. The method of claim 50, wherein said step of providing at least one polymer further comprises a step of providing at least one polymer that contains at least one functional group chosen from the group consisting of SO₃H and H₂N(CH₃).

54. The method of claim 50, further comprising a step of providing two or more charged polymers chosen from the group consisting of two-dimensional charged polymers and three-dimensional (3D) charged polymers, each of which of said charged polymers comprises materials containing cationic and/or anionic groups capable of dissociation and spatially organized in a manner adapted to preserve the pH of said body fluid according to preset conditions; said spatial organization chosen from the group consisting of (a) interlacing; (b) overlapping; (c) conjugating; (d) homogeneously mixing; (e) heterogeneously mixing; and (f) tiling.

55. The method of claim 54, further comprising a step of spatially organizing each of said functional groups in a manner selected from (a) interlacing; (b) overlapping; (c) conjugating; (d) homogeneously mixing; (e) heterogeneously mixing; and (f) any combination of the above.

56. The method of claim 50, further comprising an additional step of providing said charged polymer with an ionomeric barrier layer comprising a sulfonated tetrafluoroethylene copolymer, said barrier adapted to avoid heavy ion diffusion.

57. A method of production of a medical device effective for killing cells, comprising the steps of:
a. providing at least one charged polymer, said at least one charged polymer characterized, when in contact with a body fluid, as:
i. carrying strongly acid and/or strongly basic functional groups;
ii. having a pH of less than about 4.5 or greater than about 8.0;
iii. capable of generating an electrical potential within the confined volume of said cell sufficient to disrupt effectively the pH homeostasis and/or electrical balance within said confined volume of said cell; and,
iv. being in a form chosen from the group consisting of (i) H⁺ and (ii) OH⁻; and,
b. incorporating said charged polymer onto at least one surface of a medical device.

58. The method of claim 57, wherein said step of providing at least one electrolyte charged polymer characterized, when in contact with said body fluid, by at least one characteristic chosen from the group consisting of (a) sufficiently water-insoluble such that at least 99% remains undissolved at equilibrium; (b) sufficiently resistant to leaching such that the total concentration of material leached from said composition of matter into said body fluid does not exceed 1 ppm; (c) sufficiently inert such that at least one parameter of said body fluid chosen from the group consisting of (i) concentration of at least one predetermined water-soluble substance; (ii) particle size distribution; (iii) rheology; (iv) toxicity; (v) color; (vi) taste; (vii) smell; and (viii) texture remains unaffected according to preset conditions, said conditions adapted for and appropriate to said particular environment.

59. The method of claim 57, wherein said step of providing at least one charged polymer further comprises the step of providing a charged polymer characterized, when in contact with said body fluid, as being sufficiently inert such that the toxicity of said body fluid as defined by at least one parameter chosen from the group consisting of (a) LD₅₀ and (b) ICT₅₀ remains unaffected according to preset conditions, said conditions adapted for and appropriate to said particular environment.

60. The method of claim 57, further comprising steps of:
c. providing at least one external proton-permeable surface with a predetermined functionality; and
d. layering at least a portion of said proton-permeable surface with at least one of said charged polymer.

61. The method of claim 57, wherein said step of providing at least one polymer further comprises a step of providing at least one polymer chosen from the group consisting of (a) polyvinyl alcohol; (b) polystyrene sulfonate; and (c) polypropylene polystyrene-divinylbenzene.

62. The method of claim 57, wherein said step of providing at least one polymer that contains at least one functional group chosen from the group consisting of SO₃H and H₂N(CH₃).

63. A method for regenerating the biocidic properties of a medical device as defined in claim 37, said method comprising at least one step chosen from the group consisting of (a) regenerating said medical device's proton absorbing and/or releasing capacity; (b) regenerating said medical device's buffering capacity; and (c) regenerating the proton conductivity of said medical device.
